(19) United States
(12) Patent Application Publication
(10) Pub. No.: US 2006/0254597 A1
(43) Pub. Date: Nov. 16, 2006

(54) Method of treatment of atrophic vaginitis by topical clitoral menthol or a related cooling compound

(75) Inventor: Ronald J. Thompson, Fort Thomas, KY (US)

Correspondence Address: Donald N. Halgren, 35 Central St, Manchester, MA, 01944, US

(73) Assignee: 40J's LLC

(21) Appl. No.: 11/483,324

(22) Filed: Jul. 7, 2006

Related U.S. Application Data
(63) Continuation-in-part of application No. 11/407,383, filed on Apr. 19, 2006.
(63) Continuation-in-part of application No. 11/174,037, filed on Jul. 1, 2005.
(63) Continuation-in-part of application No. 09/736,973, filed on Dec. 14, 2000, now abandoned.

(51) Int. Cl. A61F 6/04

(52) U.S. Cl. 128/844

(57) ABSTRACT

Atrophic vaginitis is the pathologic thinning of the vaginal mucosa caused by the lack of adequate estrogen in menopause, perimenopause, or by contraceptive medications. Historically, treatment has been the replacement of estrogen, either systemically or locally, applied to the vaginal mucosa. Intravaginally applied tissue moisturizers have proven as effective as estrogen in therapy for atrophic vaginitis. We hereby describe a treatment of atrophic vaginitis that is non-hormonal and does not require intravaginal application, but rather relies upon the production of a natural vaginal serum transudate. This natural vaginal serum transudate lubrication is reflexly invoked by the daily external stimulation of the clitoral nociceptors, to treat atrophic vaginitis.

Cross-reference is made to prior applications and is a continuation in part of U.S. patent application Ser. No. 11/407,383, filed Apr. 19, 2006 and is a continuation-in-part of Ser. No. 11/174,037 filed Jul. 1, 2005, and is a continuation-in-part application of my earlier pending U.S. patent application Ser. No. 09/736,973, filed Dec. 14, 2000, entitled “Clitoral Sensitizing Arrangement,” and a continuation-in-part application of U.S. Pat. No. 6,322,493, entitled “Expanded Clitoral Sensitizing Compounds with Methods and Apparatus for the Delivery of these Compounds,” all of which are incorporated herein by reference in their entirety.

### BACKGROUND OF THE INVENTION

1. Field of the Invention

This invention relates to the treatment of atrophic vaginitis by the topical application of a compound of menthol and/or a related cooling compound applied to the clitoris of a female.

2. Prior Art

Estrogens, with the predominant estrogen, estradiol, are produced in the pre-menopausal ovary. A woman's estrus cycle is divided into the Follicular phase, the first fourteen days of the cycle, and the Luteal phase, the last fourteen days of the cycle. During the follicular phase, an ovum is actively developing in the ovarian follicle and estrogen exclusively is produced. During the Luteal phase the ovary produces predominantly progesterone, but also some estrogens. The defining event between the Follicular phase and the Luteal phase is ovulation, the release of ovum from the ovarian follicle.

As sex hormones, estrogens are produced in one tissue, the ovary. They are released into the bloodstream, and have their effect on target tissues. Those target tissues have receptors specific for recognition of the estrogen. The estrogens effect on the target tissue is to produce specific tissue growth. Target tissues for estrogen include breast tissue, vaginal tissue, vulvar tissue, endometrial, and uterine tissue. Other tissues reported to be target tissues for estrogen include bone, brain, and cardiovascular tissue.

Young women who are not on contraceptive medications have a very high level of estrogen, for the intent of contraceptives is to suppress normal ovarian function and prevent ovulation, therefore preventing a possible pregnancy. As each woman normally ages, her level of estrogen declines yearly until menopause, where the ovary is depleted of all ovum, and all estrogen production ceases. “Perimenopause” is the ill-defined time frame before and after menopause. Perimenopause could more appropriately be termed “premenopause,” for the estrogen levels for five to ten years before actual menopause (normally age 50) causes a host of signs and symptoms of inadequate estrogen in its target tissues. Perimenopause depression could be inadequate estrogen for its target brain tissue. Osteoporosis is the thinning of trabecular bone associated with genetic predisposition, low calcium intake, inadequate exercise, and low estrogen levels.

The most well recognized sequel of inadequate serum estrogen in both menopausal and perimenopausal women is the lack of adequate stimulation of the target urogenital tissue. These tissues include the vagina, vulva, urethra, and uterus. Atrophic vulvitis is the “thinning” of vulvar tissues, including the clitoris, clitoral hood, vestibule, labia minora, and labia major. Normally well estrongenized young women have a cell thickness of twenty to twenty five cells, with the cells each being “plump” and normal. Atrophic vulvitis is diagnosed by the cell thickness being only eight to ten cells thick, where each cell is thin and flat. Bioassays to determine estrogen effectiveness have been performed in castrated rats or rabbits, wherein different estrogens are administered daily for several weeks. A vulvar or a vaginal lavage during such a bioassay is then placed into a glass slide for microscopic examination. A few sparse flat cells indicate a low estrogen effect whereas numerous plump cells indicate a high estrogen effect.

Hormone Replacement Therapy (HRT) has been known for many years. Historically, estrogens as HRT have been used for decades, if not centuries, to treat the vasomotor symptoms (hot flashes, night sweats, and depression) of inadequate estrogen production associated with menopause. This treatment of estrogen replacement therapy only needs one to two years of duration, for the women's body will accommodate to the high levels of Follicle Stimulating Hormone (FSH), produced by the pituitary gland in the attempts to induce the ovaries to produce estrogens.

In the 1970's, attempts to extend the estrogen mediated youthfulness beyond age 50 (menopause), and for women who had their ovaries surgically removed in their thirties or forties, standard therapy was estrogen replacement. This estrogen replacement treatment was intended to continue for ten, twenty, thirty, or even forty years. The theory behind prolonged estrogen replacement therapy was the positive impact on the estrogen target tissues. Estrogen will maintain youthfulness of the vulvar and vaginal tissues by stimulating tissue growth and preventing atrophic vulvitis or atrophic vaginitis caused by inadequate estrogen levels.

A second and more theoretical benefit of prolonged estrogen therapy was to prevent osteoporosis, cardiovascular disease, and even brain disorders such as dementia or cerebral atrophy. The prolonged estrogen therapy also necessitated the intermittent use of progesterone to prevent unopposed stimulation of the endometrium. Use of estrogen and progesterone was patterned after the normal Follicular/Luteal phases with estrogen given for twenty five days of each calendar month, and progesterone given for the last ten days of estrogen therapy. After the progesterone is stopped, the patient will have a progesterone-withdraw menstrual period, and sometimes this will not be accepted by women in their sixties and seventies!

There are various types of Estrogen, its routes of administration, and the use of pharmakenetics. “Systemic” in pharmacokenetics means distributed through the entire body system and available to all cells of the body. The predominant normal estrogens produced by the ovaries, estrone, estradiol, and esteriol are only lipid soluble, and therefore not absorbed from the stomach or the intestines. Estradiol is absorbed if injected into the muscle, transdermally absorbed from an “estrogen patch” worn on the stratified squamous skin of the abdomen or buttocks, or applied to the mucous membrane of the vagina or the non-Keratinized epidermis of the vulva. Micronized estradiol orally ingested is partially absorbed from the stomach and the intestine mucosa. Estrogen salts, with the most commonly prescribed being PREMARIN, (pregnant mare urine) are conjugated estrogens that are readily absorbed from the stomach and intestinal mucosa, and are therefore orally active estrogens. A PREMARIN cream is likewise easily absorbed from the vaginal and vulvar tissues.

Estradiol injections or transdermal patches of estradiol provide a constant availability of estradiol in the blood stream. Oral Premarin or oral micronized estradiol provides a large amount of estrogen to the blood stream initially, but this “adequate estrogen” can decay in twelve to twenty four hours. Some patients require oral estrogens every twelve hours (twice per day dosing) to prevent the hot flashes and night sweats. Topical vaginal or vulvar estrogens are effective in the vulvae/vaginal tissue to treat or prevent atrophic vulvitis/vaginitis of inadequate estrogen with only once/day application. The topical vaginal/vulvar estrogen creams are ineffective in treatment of hot flashes night sweats, or insomnia, and are therefore not effective systemically, even if applied several times per day.

Estrogen has implications for breast tissue and breast cancer. Breast tissue is a “target tissue” for estrogen. Breast tissue is also a target tissue for progesterone. Upon removal, breast cancer is analyzed for the presence of estrogen and progesterone receptors. Cancer is the unregulated growth of any tissue. While multiple studies have shown variable associations between breast cancer and estrogen, some show a direct cause and effect relationship, and others showing no cause and effect relationship. The unclear cause and effect relationship between the use of systemic estrogen long-term and the development of breast cancer causes more and more women to decline or abandon estrogen therapy. This decline of the use of HRT has increased the incidence of atrophic vulvitis/vaginitis because of the absolute lack of estrogen. Because of the generalized fear of estrogens, women are even reluctant to use topically active, non-systemic, estrogens to prevent or treat atrophic vulvitis/vaginitis.

Topical intravaginal estrogen is very effective in the treatment of atrophic vaginitis. Dr. Ballagh in “Vaginal hormone therapy for urogenital and menopausal symptoms,” published in the May 2005 Seminars in Reproductive Medicine reports that “Urogenital symptoms that clearly respond to estrogen therapy include atrophic vaginitis, dryness and accompanying dyspareunia.” He also reports that “even the lowest dose estradiol (7.5 mg daily or 25 mg twice per week) shows evidence of systemic absorption.” Dr. Samsione concluded in his 1998 article “Urogenital aging—A hidden problem,” (American Journal of Obstetrics and Gynecology, May 1998) that “The systemic absorption of low-dose estrogen preparations is dependent on the status of the vaginal mucosa. Absorption is high when the vaginal mucosa is atrophic and gradually decreases (but not to zero) as the vaginal mucosa matures under estrogen influence.”

During the Women's Health Initiative, over 68,000 postmenopausal women were to be studied over a 15 year period to determine the risks and benefits of hormone therapy. Women were administered estrogen alone, estrogen plus progesterone, and placebo. Women on estrogen alone were informed in March 2004 that while estrogen replacement therapy may have some positive effects in the treatment of menopausal symptoms, estrogen therapy increases the risk of breast cancer. The Women's Health Initiative study was halted after this announcement in March of 2004 and further results have been reported through 2005. Estrogen was found to increase the risks of stroke, vein thrombosis, and may have increased the risks of breast cancer. Estrogen and progesterone administered at the same time were found to increase the risk of breast cancer by 24%.

Alternative Therapy for Atrophic Vaginitis

Replens is a polycarbophil-based vaginal moisturizer. In the 1994 Fertility and Sterility article “Comparative Study: Replens verses local estrogen in menopausal women,” Dr. Nachtigall concludes “Results indicated that the bioadhesive vaginal moisturizer was a safe and effective alternative to estrogen vaginal cream, with both therapies exhibiting statistically significant increases in vaginal moisture, vaginal fluid volume, and vaginal elasticity with a return of the premenopausal pH state.” In a subsequent article, “Replens verses dienoesterol cream in the symptomatic treatment of vaginal atrophy in postmenopausal women,” Dr. Bygdeman concludes “This study shows that Replens applied vaginally three times a week, is a full therapy for all symptoms of vaginal atrophy as well as local estrogen.” Dr. Bygdeman's article was published in Maturitas, 1996 April.

Both articles cited conclude that Replens is as effective as topical estrogen for the treatment of atrophic vaginitis, but without the risks of estrogen! The Replens must be inserted into the vagina three times per week to achieve an adequate level of effectiveness for treatment of atrophic vaginitis. Dr. Crandall reported in the December 2002 Journal of Women's Health, “Vaginal estrogen preparations: a review of safety and efficacy for vaginal atrophy” that “Nonhormonal lubricant is effective in improving some atrophic signs and symptoms. All preparations were associated with vaginal irritation.”.

### BRIEF DESCRIPTION OF THE DRAWINGS

The objects and advantages of the present invention will become more apparent when viewed in conjunction with the following drawings which are labeled with the proper biological terms to facilitate appreciation and ease of comprehension, in which:

FIG. 1 represents a sectional view of a normal vaginal mucosa;

FIG. 2 represents a sectional view of vaginal mucosa tissue showing atrophic vaginitis;

FIG. 3 represents a sectional view of vaginal mucosa tissue with atrophic vaginitis treated with estrogen;

FIG. 4 represents a sectional view of vaginal mucosa tissue with atrophic vaginitis treated with Replens, a cellular moisturized;

FIG. 5 represents a sectional view of vaginal mucosa tissue with atrophic vaginitis after two weeks of treatment with topical clitoral menthol;

FIG. 6 represents a sectional view of vaginal mucosa tissue with atrophic vaginitis after six weeks of treatment with topical clitoral menthol; and

FIG. 7 represents a portion of the female reproductive and nervous system.

### DESCRIPTION OF THE PRESENT INVENTION

Our previous U.S. patent application Ser. No. 11/174,037, incorporated herein by reference in its entirety, and entitled “Topical Menthol or a Related Cooling Compound to Induce Lubrication” describes a reflex vaginal transudate lubrication in response to the application of menthol to the clitoris. The invention described in U.S. patent application Ser. No. 11/174,037 is however, specifically adapted to induce sexual lubrication used on an episodic schedule with sex and intimacy.

However, women aged 35 to 60 reported better episodic or intermittent effectiveness of the topical menthol & L-arginine if they used the product daily as well as with intercourse. Therefore, to increase the effectiveness of the episodic use we changed the directions for use to daily application of the menthol and L-arginine product. Surprisingly, the women aged 35 to 60 who used the topical menthol and L-arginine on a daily basis after a shower or bath reported less symptoms of vaginal irritation and dryness after several weeks of use. The only explanation for this effect is the physiologic therapy for the atrophic vaginitis by the stimulation of daily vaginal lubrication. Therefore, it was unanticipated that the daily use of topical clitoral menthol and L-arginine could be as effective therapy for atrophic vaginitis caused by age related estrogen decrease.

Therefore, we now hereby describe the use of topical clitoral menthol as a regular, preferably daily basis, to induce the daily vaginal transudate to un-anticipatedly treat atrophic vaginitis, surprisingly without topical/intravaginal estrogen, and without topical intravaginal moisturizers.

The invention thus comprises a topical clitoral menthol compound for inducing transudate lubrication, which compound could be delivered by manual digital application, a foam spray from a hand manipulable pressurizable foam or spray applicator, or even in a tissue-adherable matrix dissolvable sheet.

The invention also comprises a method for a non-hormonal treatment of atrophic vaginitis to stimulate the production of a natural vaginal serum transudate, comprising a compound of menthol for application to the vagina and clitoris.

The invention also comprises a method of effecting natural vaginal serum transudate lubrication by reflexly invoked external stimulation of the clitoral nociceptors, by one or more of the following the steps of: applying a compound of topical clitoral menthol to the vaginal tissue for the therapeutic treatment of atrophic vaginitis, wherein the topical clitoral menthol is delivered by manual digital application; or wherein the topical clitoral menthol is delivered by a foam from a hand manipulable pressurizable foam applicator; or wherein the topical clitoral menthol is delivered by a spray from a hand manipulable pressurizable spray applicator; or wherein the topical clitoral menthol is delivered by a body tissue-adherable matrix of menthol cooling compound carried in a dissolvable sheet.

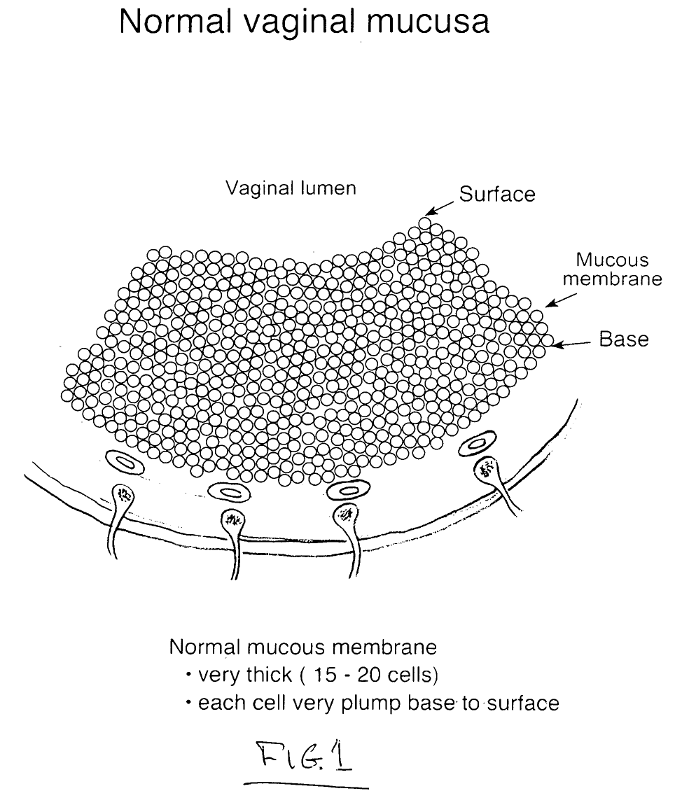

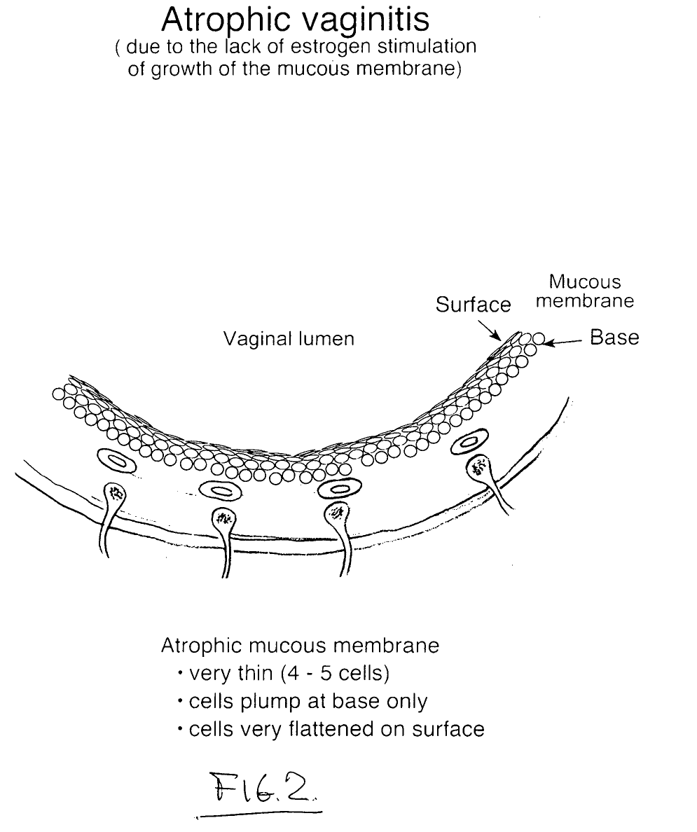

For example, in the drawings, FIG. 1 represents a sectional view of a normal vaginal mucosa. The vaginal lumen is surrounded by a thick, highly populated mucous membrane with an outer base layer. Such normal mucous membrane may be about 15 to 20 cells thick. Each cell being very plump, from base layer to the inner surface layer. FIG. 2 represents a sectional view of vaginal mucosa tissue showing atrophic vaginitis, with a very thin mucous membrane, that is, about 4 to 5 cells thick, with cells plump at only the base layer. The cells on the surface of the vaginal lumen are very flattened. This is due to the lack of estrogen stimulation of growth of the mucous membrane.

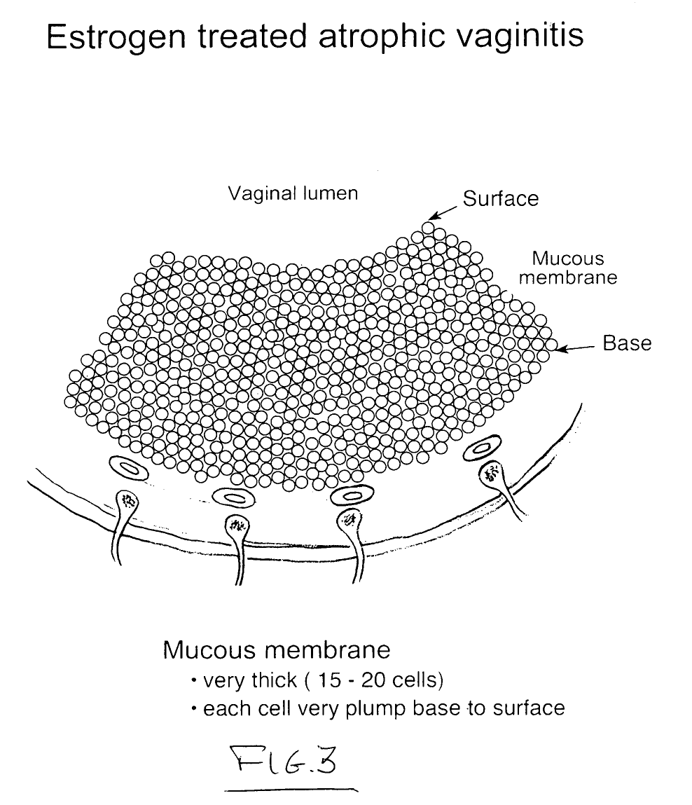

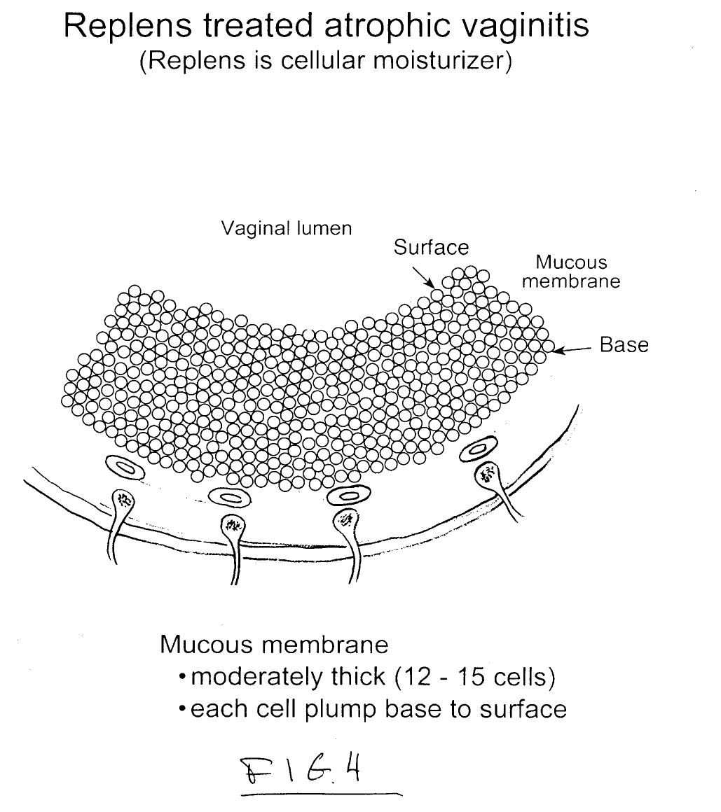

FIG. 3 however, represents a sectional view of vaginal mucosa tissue with atrophic vaginitis treated with estrogen. The mucous membrane is very thick, some 15 to 20 cells thick. Each cell is also very plump, from the base layer to the surface layer adjacent the vaginal lumen. FIG. 4 represents a sectional view of vaginal mucosa tissue with atrophic vaginitis treated with Replens, a cellular moisturized. It however, is only moderately thick, some 12 to 15 cells thick, with each cell plump from the base layer to the surface layer.

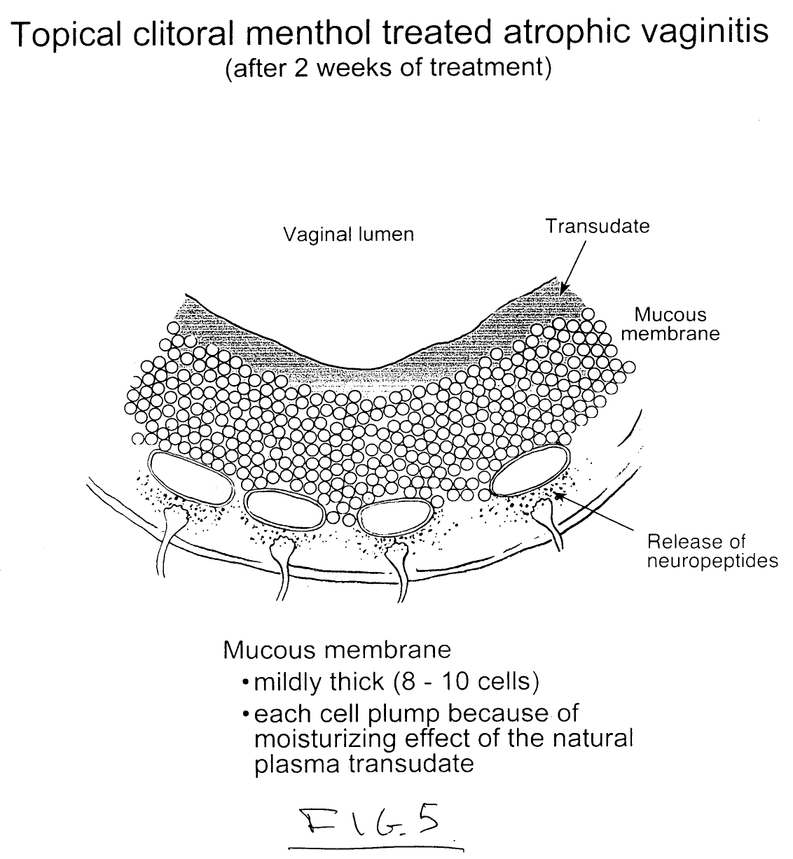

Surprisingly however, a topical treatment with menthol on the atrophic vaginitis, represented in FIG. 5 in a sectional view, shows vaginal mucosa tissue with atrophic vaginitis after only a mere two weeks of treatment with that topical clitoral menthol. The mucous membrane has become at least mildly thick, with some 8 to 10 cells therein, each cell being plump because of the surprising moisturizing effect of the natural plasma transudate and the release of neuropeptides within the cell structure.

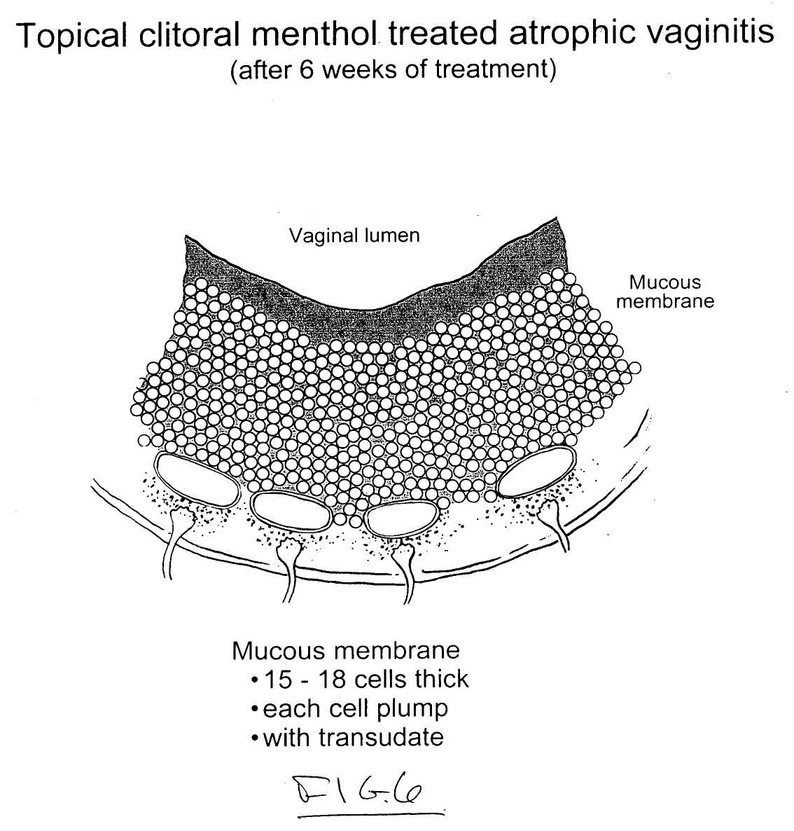

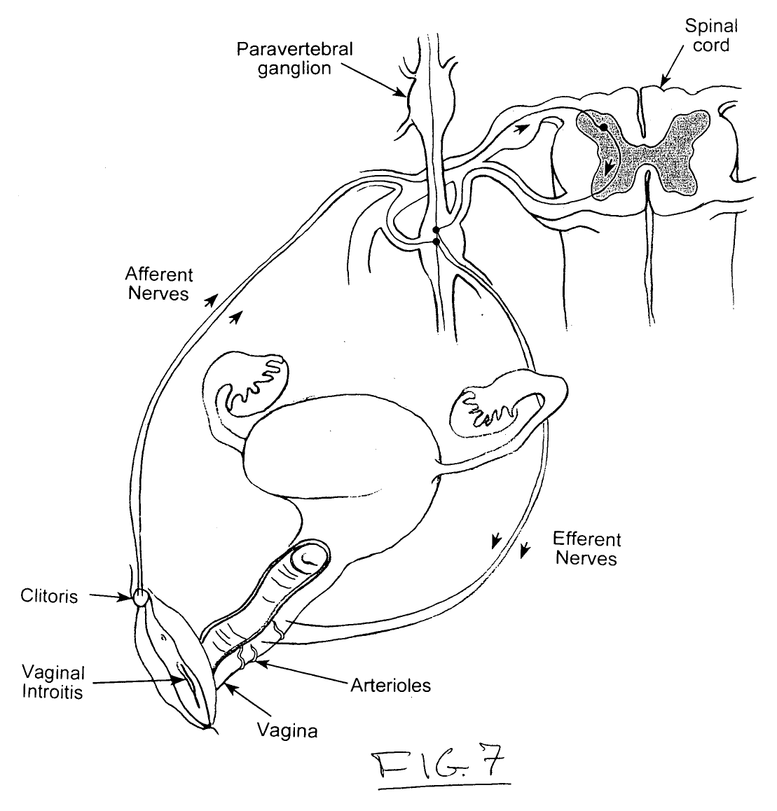

FIG. 6 represents a sectional view of vaginal mucosa tissue which had atrophic vaginitis, and which surprisingly and un-anticipatedly improved markedly after only six weeks of treatment with topical clitoral menthol. The mucous membrane is now some normal 15 to 18 cells thick, each cell being plump, with ample transudate. FIG. 7 represents a portion of the female reproductive and nervous system.

I claim:

1. A non-hormonal treatment of atrophic vaginitis to stimulate the production of a natural vaginal serum transudate, comprising a compound of menthol for application to the vagina and clitoris.

2. A method of effecting natural vaginal serum transudate lubrication by reflexedly invoked external stimulation of the clitoral nociceptors, by:
applying a compound of topical clitoral menthol to the vaginal tissue for the therapeutic treatment of atrophic vaginitis.

3. The method as recited in claim 2, wherein said topical clitoral menthol is delivered by manual digital application.

4. The method as recited in claim 2, wherein said topical clitoral menthol is delivered by a foam from a hand manipulable pressurizable foam applicator.

5. The method as recited in claim 2, wherein said topical clitoral menthol is delivered by a spray from a hand manipulable pressurizable spray applicator.

6. The method as rected in claim 2, wherein said topical clitoral menthol is delivered by a body tissue-adherable matrix of menthol cooling compound carried in a dissolvable sheet.

7. The method as recited in claim 2, wherein said application of topical menthol is applied to the vaginal tissue on a daily basis.

8. The method as recited in claim 1, wherein said compound includes a composition of L-arginine.
